(19) United States
(12) Patent Application Publication
(10) Pub. No.: US 2025/0221817 A1
(43) Pub. Date: Jul. 10, 2025

(54) OPHTHALMOLOGICAL COMPOSITION HAVING A PLURALITY OF COMONOMER GROUPS, AND OPHTHALMOLOGICAL LENS

(71) Applicant: Carl Zeiss Meditec AG, Jena (DE)

(72) Inventors: Thorben Badur, Berlin (DE); Stephen Zhou, Irvine, CA (US); Minh Cao, Santa Ana, CA (US)

(21) Appl. No.: 19/094,570

(22) Filed: Mar. 28, 2025

Related U.S. Application Data
(63) Continuation of application No. PCT/EP2023/075777, filed on Sep. 19, 2023.

(30) Foreign Application Priority Data
Sep. 30, 2022 (DE) 10 2022 125 341.4

(51) Int. Cl. A61F 2/16; A61L 27/16

(52) U.S. Cl. CPC A61F 2/16; A61L 27/16; A61F 2002/1699

(57) ABSTRACT

Ophthalmological compositions for producing ophthalmological lenses include comonomer groups A) to C), at least one comonomer group which is D), E), or a mixture thereof, and at least one crosslinker F). Here: A) denotes at least one (meth)acrylate having at least one aromatic group; B) denotes at least one (meth)acrylate having an aliphatic or non-aromatic cyclic or non-aromatic heterocyclic group; C) denotes at least one (meth)acrylate having at least one hydroxyl group; D) denotes at least one (meth)acrylamide having an aromatic group and having an aliphatic or non-aromatic cyclic or non-aromatic heterocyclic group; and E) denotes a mixture of at least one (meth)acrylamide having two aromatic groups and at least one (meth)acrylamide having two aliphatic and/or non-aromatic cyclic and/or non-aromatic heterocyclic groups. Ophthalmological lenses are produced at least in part from the described ophthalmological compositions.

### CROSS-REFERENCE TO RELATED APPLICATIONS

This application is a continuation application of international patent application PCT/EP2023/075777, filed Sep. 19, 2023, designating the United States and claiming priority from German application 10 2022 125 341.4, filed Sep. 30, 2022, and the entire content of both applications is incorporated herein by reference

### TECHNICAL FIELD

Disclosed is an ophthalmological composition including two or more comonomer groups and an ophthalmological lens, including a soft intraocular lens, that has been produced at least partly from such an ophthalmological composition.

In recent decades, a wide range of different biomaterials have been developed that are suitable for production of ophthalmological compositions and lenses, especially intraocular lenses (IOL). The different classes of materials include hydrophilic polymers, hydrophobic polymers and silicone. Each class has its own advantages and disadvantages. Although silicone IOLs have very good resistance to posterior capsule opacity (PCO), their unfolding behavior after injection can be uncontrollable. Hydrophilic lenses generally show very good biocompatibility, but also higher PCO and calcification rates. Hydrophobic IOLs have recorded the greatest growth in recent years. They generally offer a relatively high refractive index in the range of n_{D}=1.44-1.55, but harbor the risk of “glistening”, which is characterized by aqueous microvacuoles which form within the polymer matrix and which can disrupt the patient's visual perception, especially with multifocal lenses. Vacuoles with diameters of less than 200 nm that are up to 120 μm beneath the surface of the IOL are also referred to as “nanoglistening” (subsurface nanoglistenings, SSNG).

US 2002/0049290 A1 discloses optically transparent hydrogels with a high refractive index and intraocular lenses made therefrom. The preferred hydrogels have a refractive index of 1.45 or more and a water content of about 5 to 30 percent by weight.

WO 99/58507 A1 discloses hydrophilic, UV light-absorbing, polymerizing monomers. These monomers are copolymerizable and give rise to biocompatible hydrogels that can absorb at least 90% of the UV light incident on the hydrogels. Such hydrogels are optically transparent, have high refractive indices and have long-term stability.

US 2013/0231740 A1 discloses relatively soft, optically transparent, foldable materials with a high refractive index, which are particularly suitable for use in the manufacture of intraocular lenses, contact lenses and other eye implants.

In order to tailor a material to such a specific application as an ophthalmic lens, complex multiparameter optimization has to be performed. In addition to ensuring biocompatibility, optical, physical, and mechanical properties also have to be optimized at the same time. For example, a material with excellent biocompatibility, but at the same time with low flexibility, is not ideal, since this would lead to larger incisions for the implantation of the IOL. This is particularly important because the aim is to make ever smaller incisions (microincision cataract surgery, MICS with <2 mm incision). The selection of the base material is particularly important. Hydrophobic polymers intrinsically offer good base properties for the development of an MICS-compatible material, namely high tensile strength in combination with a high refractive index. However, glistening resistance and material flexibility have to be significantly increased. A new class of materials, called “new hydrophobic acrylates,” was the result of recent research in the field of IOL materials. An overview of the currently standard material classes is given in table 1 below.

TABLE 1
Physical properties of standard IOL materials

| | | Contact | Tensile | | |
|---|---|---|---|---|---|
| | Hyg. | angle | strength | Refractive | |
| Material | (%) | (°) | (MPa) | index n | T_{g} (° C.) |
| PMMA | 0.4-0.8 | 65-71 | 47-70 | 1.49 | 105-113 |
| Silicone | 0.38 | 97-120 | 5.9-8.2 | 1.43 | −120 to −90 |
| Hydrophilic | 18-33 | 20-70 | 0.4-0.6 | 1.40-1.43 | 10-20 |
| acrylates | | | | | |
| Hydrophobic | 0.1-0.5 | 72-88 | unknown | 1.47-156 | 5-16 |
| acrylates | | | | | |
| New | about | 69-79 | 4-6 | 1.54 | 27-29 |
| hydrophobic | 4-5 | | | | |
| acrylates | | | | | |

In Table 1, “Hyg.” denotes the percentage by mass of water in the lens at equilibrium at 35° C., “contact angle” is the angle between the leading edge of a water drop and the material surface, “tensile strength” is the greatest stress that the material class can withstand while being drawn without breaking, “refractive index n” denotes the refractive index at 20° C., and “Tg” denotes the glass transition temperature.

Hydrophilic materials generally have a low refractive index, which actually decreases further when they are fully hydrated. Therefore, lens curvature and thickness must automatically be more significant at high diopters than in the case of materials having a higher refractive index. This requires the implantation of a large lens cross section through a small injection tip, which increases the risk of damage to the cartridge tip of the implantation tool or the lens itself. Higher material flexibility can be achieved in the case of these materials via a high water absorption of 5 to 30 percent. However, not only does this significantly reduce the refractive index, but also entails storage of the lenses in an aqueous or at least moisture-controlled packaging. Otherwise, it is not possible to assure dimensional accuracy and optical quality after lens implantation.

The main idea of the “new hydrophobic acrylates” is to add the hydrophilic monomer HEMA (2-hydroxyethyl methacrylate) to otherwise hydrophobic comonomers, since this has the ability to disperse water throughout the material. Known ophthalmological compositions contain about 30% HEMA in order to be free of glistening, and about 4% by weight of water at equilibrium. Because of the latter proportion, corresponding IOLs have to be stored in a 0.9% saline solution, and are therefore not suitable for dry-preloaded IOL implantation systems.

In the case of some “new hydrophobic acrylates,” the risk of glistening was supposedly reduced, but at the cost of a relatively low refractive index n_{d}<1.5. This means, as already mentioned, that the cross-section of the lens and hence also the required cut size have to be significantly increased depending on the number of diopters. In addition, these materials have a relatively slow unfolding speed of the lens under simulated operating conditions (26° C. water bath) and are not suitable for steam sterilization, which severely limits the options for sterilization procedures, but these are mandatory for ophthalmological implants.

The latest developments in the field of IOL biomaterials thus point to an interesting direction for further development, but improvements are still needed toward implants that offer a faster and less invasive surgical procedure combined with higher patient satisfaction.

### SUMMARY

It is therefore described herein an ophthalmological composition that facilitates the production of flexible biocompatible ophthalmological lenses with a high refractive index coupled with the lowest possible risk of glistening, where an ophthalmological lens produced from the composition is both storable dry and sterilizable by steam sterilization. Further described herein is a corresponding ophthalmological lens.

Therefore, provided is an ophthalmological composition possessing the features recited in claim 1 and an ophthalmological lens according to claim 6. Advantageous configurations of the described lens are specified in the respective dependent claims; advantageous configurations of the ophthalmological composition are to be regarded herein as advantageous configurations of the described ophthalmological lens, and vice versa.

Described herein is an ophthalmological composition for production of an ophthalmological lens, including comonomer groups A) to C), at least one comonomer group which is D), E), or a mixture thereof, and at least one crosslinker F). In this context, A) denotes at least one (meth)acrylate having at least one aromatic group; B) denotes at least one (meth)acrylate having an aliphatic or nonaromatic cyclic or nonaromatic heterocyclic group; C) denotes at least one (meth)acrylate having at least one hydroxy group; D) denotes at least one (meth)acrylamide having an aromatic group and having an aliphatic or nonaromatic cyclic or nonaromatic heterocyclic group; and E) denotes a mixture of at least one (meth)acrylamide having two aromatic groups and at least one (meth)acrylamide having two aliphatic and/or nonaromatic cyclic and/or nonaromatic heterocyclic groups. In other words, it is envisaged in accordance with the invention that the ophthalmological composition includes monomers from at least four or five different molecular classes A), B), C), D), or A), B), C), E), or A), B), C), D), E) and at least one crosslinker F), or in the simplest form consists of the molecular classes mentioned and does not contain any other molecular classes. The compositions described herein are in some embodiments silicon-free and/or siloxane-free and in others free of fluorinated compounds. The molecular classes A) to E) act as comonomers in the synthesis of a polymer from the ophthalmological composition of the invention, and can be crosslinked with the aid of the crosslinker F). The compositions described herein are also referred to as prepolymers. The term “(meth)acrylate” used in the context herein refers to acrylates, methacrylates, and any mixtures thereof, in the absence of any explicit discussion of particular individual compounds. For example, the expression “2-phenylethyl (meth)acrylate” is contemplated to encompass the compounds 2-phenylethyl acrylate, 2-phenylethyl methacrylate, and any mixtures of these two compounds. The same applies to the expression “(meth)acrylamide,” which encompasses acrylamide, methacrylamide, or a mixture thereof, in the absence of any explicit discussion of particular individual compounds. In general, “a/an” in the context of this disclosure is to be read as the indefinite article, that is, always as “at least one” in the absence of any express indication of the contrary. Conversely, “a/an” also is to be understood to mean “just one.”

The ophthalmological composition described herein is suitable in particular for production of soft, MICS-compatible intraocular lenses, but are also used in principle for production of other ophthalmological lenses, implants, keratoprostheses, inlays, and the like. At the same time, the compositions described herein allow the production of ophthalmological lenses that provide high patient satisfaction because of their very low tendency of glistening. The surgical procedure in an implantation is therefore additionally improved by the fast, controllable unfolding speed of the lenses made from the described compositions. The ophthalmological compositions described herein also allows for the production of ophthalmological lenses that are in some instances used in fully preloaded injector systems with dry and wet storage. This reduces manufacturing complexity and facilitates storage while maintaining full design freedom. Finally, the ophthalmological compositions described herein or a lens produced therefrom are, in some embodiments, sterilized by steam sterilization. This not only opens up a wider range of production processes but also the use of a more sustainable and less costly method compared to the established ethylene oxide (EtO) sterilization of standard hydrophobic IOLs.

The present disclosure is based on the finding that, in order to design a polymeric biomaterial that incorporates all the above-mentioned requirements if at all possible, what is required is an ophthalmological composition including at least four different classes of monomers or comonomers, each with different properties, as explained in further detail hereinbelow.

The first group A) may also be referred to as “aromatic monomers,” which include at least one aromatic group or an aryl ring in order to increase the refractive index of the resulting polymer. The term “aryl ring” includes both individual rings, for example phenyl, and fused, and isolated aromatic ring systems, for example naphthyl or biphenyl rings. The aryl ring(s), in some embodiments, bear one or more substituents. The aryl ring is in some embodiments selected from C₆₋₁₈-aryl groups.

The second group B) includes monomers possessing an aliphatic group, which is used to increase the flexibility of the material. Alternatively or additionally to an open-chain aliphatic group, in some embodiments, one or more nonaromatic cyclic groups, for example a cyclohexyl group, and/or one or more nonaromatic heterocyclic groups, for example a piperidinyl group, to be provided. The aliphatic group is in some embodiments selected from C₁₋₁₂alkyl groups that are in some embodiments unbranched or—to the extent possible—branched. The heterocyclic group in some embodiments includes at least one atom from the group of N, S, and/or O and is suitable for the formation of hydrogen bonds. The aliphatic group(s) are in some embodiments linear, but are in principle also able to be branched. Furthermore, each aliphatic nonaromatic cyclic or nonaromatic heterocyclic group also in some embodiments include one or more carbon double and/or triple bonds.

The third group C) includes one or more monomers including at least one hydroxy group. These are in some embodiments one or more terminal or sterically unhindered hydroxy groups that is/are capable of forming hydrogen bonds in the polymer. The third group C) is added in order to increase the water absorption of the polymer composition.

The fourth group D) includes “hybrid” (meth)acrylamide comonomers that lead to a surprising improvement in the resulting biomaterial properties. The molecular class D), defined herein as “hybrid” (meth)acrylamide comonomers, includes a vinyl functionality for polymerization and a tertiary amide. The advantage of incorporating an acrylamide over an acrylate is that two terminal substituents are in some embodiments attached per monomer unit. In the case of the “hybrid” acrylamides, the two substituents or functional groups are different in group D). A comonomer of group D) thus has the general formula (I):

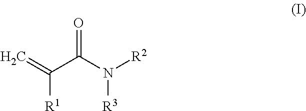

in which R¹═H/CH₃, R²=aryl-containing radical and R³=alkyl radical, where R² and/or R³ are in some embodiments unsubstituted. The two substituents R² and R³ are selected such that they in some embodiments have the same or very similar chemical structures to those of the aromatic monomer A) or of the aliphatic monomer B) that are incorporated into the ophthalmological composition described herein. Accordingly, in some embodiments the same considerations and limitations apply to the substituents of the comonomer of group D) as to the substituents of the comonomers of groups A) and B). These structural similarities result in advantageous intermolecular interactions between the different comonomer groups A), B), and D) in the later polymer, in that, without wishing to be bound by theory, the acrylamide D) acts as a kind of mediator between groups A) and B). it is thought that the aryl group of the acrylamide D) also advantageously leads to an increase in the refractive index and forms n-T interactions with the molecules of the aromatic monomer A), which leads to a higher structural strength of the polymer. It is believed that the alkyl group of the acrylamides D) increases the flexibility of the material or, in the case of branched or cyclic/heterocyclic substituents, also offers higher lightfastness and tear resistance. In certain embodiments, R3 in formula (I) is alternatively in particular substituted by one or more hydroxy groups. In this way, comonomer D) is structurally similar to comonomer C), in some embodiments, and so it is correspondingly possible to develop advantageous intermolecular interactions between groups A), C), and D).

Alternatively or additionally to group D), in some embodiments group E) is also incorporated into the described compositions. The two groups D) and E) are therefore used synonymously hereinafter. Group E) includes a mixture of at least two different (meth)acrylamides, where a first (meth)acrylamide includes two aromatic groups and a second (meth)acrylamide includes two aliphatic and/or nonaromatic cyclic and/or nonaromatic heterocyclic groups. In other words, group E) includes two molecular classes of the general formulae IIa and IIb:

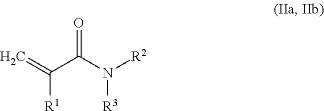

where, in formula IIa, R¹═H/CH₃ and R² and R³=aryl-containing radical and, in formula IIb, R¹═H/CH₃ and R² and R³=alkyl radical, where R² and R³ are independently unsubstituted and/or especially substituted by one or more hydroxy groups, in order to be able to interact with comonomers of group C). The two substituents R², R³ of a (meth)acrylamide are, in some embodiments, either the same, or in others are different. Otherwise, the same considerations and limitations apply to the substituents of group E) as to the substituents of groups A), B), and C). In contrast to group D), group E) thus does not encompass intramolecular “hybrid” (meth)acrylamides, but rather a “hybrid” mixture of at least two different (meth)acrylamides, where mixture E) too acts, in some embodiments, as a kind of mediator between groups A) and B) or C) in the polymer. Some of the individual compounds of group E) are, in some embodiments, synthesized more easily and in a less costly manner than the hybrid compounds of group D), especially when the individual (meth)acrylamides each bear two identical substituents R², R³.

For production of an elastomeric biocompatible polymer that is suitable for use as a soft IOL and is in some embodiments not a thermoplastic, finally, at least one crosslinker F) is provided, which is designed to create covalent bonds between polymer chains that guarantee reliable unfolding and provide a balance between material thickness and flexibility. Two or more different crosslinkers are, in some embodiments, also provided, in order in particular to adjust the mechanical properties of the polymer.

Because polymer chains regularly include areas that are denser and others that are more loosely folded, pockets with lower density can, in some instances, cause local accumulation of water, especially when environmental conditions change rapidly (for example in the event of a temperature shock). The described compositions are, therefore, also motivated by the finding that this risk is, in some instances, significantly reduced by the use of flexible substituents that are freely oriented. This is particularly true of unsubstituted or substituted alkyl groups as provided, or may be provided, in the comonomer groups B), C), D), and E).

On the other hand, it has also been recognized that it is useful to use hydrophilic or hygroscopic comonomers that are distributed in the polymer matrix with maximum homogeneity. These comonomers according to group C) not only stabilize water locally by forming hydrogen bonds but also form noncovalent bonds with the functional groups of the amide (group D)/E)), which leads to higher structural strength of the polymer.

The compositions described herein, based on the total weight of the ophthalmological composition, a proportion of comonomer group A) is, in some embodiments, between 30% by weight and 60% by weight, and so is, for example, 30% by weight, 31% by weight, 32% by weight, 33% by weight, 34% by weight, 35% by weight, 36% by weight, 37% by weight, 38% by weight, 39% by weight, 40% by weight, 41% by weight, 42% by weight, 43% by weight, 44% by weight, 45% by weight, 46% by weight, 47% by weight, 48% by weight, 49% by weight, 50% by weight, 51% by weight, 52% by weight, 53% by weight, 54% by weight, 55% by weight, 56% by weight, 57% by weight, 58% by weight, 59% by weight, or 60% by weight. A proportion of comonomer group B) is, in some embodiments, between 10% by weight and 45% by weight, and so is, for example, 10% by weight, 11% by weight, 12% by weight, 13% by weight, 14% by weight, 15% by weight, 16% by weight, 17% by weight, 18% by weight, 19% by weight, 20% by weight, 21% by weight, 22% by weight, 23% by weight, 24% by weight, 25% by weight, 26% by weight, 27% by weight, 28% by weight, 29% by weight, 30% by weight, 31% by weight, 32% by weight, 33% by weight, 34% by weight, 35% by weight, 36% by weight, 37% by weight, 38% by weight, 39% by weight, 40% by weight, 41% by weight, 42% by weight, 43% by weight, 44% by weight, or 45% by weight. A proportion of comonomer group C) is, in some embodiments, between 5% by weight and 30% by weight, and so is, for example, 5% by weight, 6% by weight, 7% by weight, 8% by weight, 9% by weight, 10% by weight, 11% by weight, 12% by weight, 13% by weight, 14% by weight, 15% by weight, 16% by weight, 17% by weight, 18% by weight, 19% by weight, 20% by weight, 21% by weight, 22% by weight, 23% by weight, 24% by weight, 25% by weight, 26% by weight, 27% by weight, 28% by weight, 29% by weight, or 30% by weight. A proportion of the sum total of comonomer groups D) and E) is, in some embodiments, between 1% by weight and 14% by weight, and so is, for example, 1% by weight, 2% by weight, 3% by weight, 4% by weight, 5% by weight, 6% by weight, 7% by weight, 8% by weight, 9% by weight, 10% by weight, 11% by weight, 12% by weight, 13% by weight, or 14% by weight. A proportion of the crosslinker F) is, in some embodiments, not more than 5% by weight, and so is, for example, 0.05% by weight, 0.1% by weight, 0.2% by weight, 0.3% by weight, 0.4% by weight, 0.5% by weight, 0.6% by weight, 0.7% by weight, 0.8% by weight, 0.9% by weight, 1.0% by weight, 1.1% by weight, 1.2% by weight, 1.3% by weight, 1.4% by weight, 1.5% by weight, 1.6% by weight, 1.7% by weight, 1.8% by weight, 1.9% by weight, 2.0% by weight, 2.1% by weight, 2.2% by weight, 2.3% by weight, 2.4% by weight, 2.5% by weight, 2.6% by weight, 2.7% by weight, 2.8% by weight, 2.9% by weight, 3.0% by weight, 3.1% by weight, 3.2% by weight, 3.3% by weight, 3.4% by weight, 3.5% by weight, 3.6% by weight, 3.7% by weight, 3.8% by weight, 3.9% by weight, 4.0% by weight, 4.1% by weight, 4.2% by weight, 4.3% by weight, 4.4% by weight, 4.5% by weight, 4.6% by weight, 4.7% by weight, 4.8% by weight, 4.9% by weight, or 5.0% by weight. The proportion of the crosslinker F) is in a particular embodiment between 0.5% by weight and 5% by weight.

The (meth)acrylamide comonomers D)/E) are used in some embodiments as a kind of “additive” in ophthalmological compositions. This means that the sum total of the (meth)acrylamides D)/E) in % by weight is preferably the smallest compared to the remaining comonomer types A) to C) in the composition. It will be appreciated that the proportions of all components of the ophthalmological composition always and exclusively add up to 100% by weight. In general, percentages in the context of the present disclosure are to be considered to be percentages by mass, unless stated otherwise.

In an advantageous configuration of the compositions described herein, comonomer group A) includes or is 2-phenylethyl acrylate, 2-phenylethyl methacrylate, ethylene glycol phenyl ether acrylate, ethylene glycol phenyl ether methacrylate, or a mixture thereof. Alternatively or additionally, comonomer group B) includes or is butyl acrylate, butyl methacrylate, isobutyl acrylate, isobutyl methacrylate, 2-ethylhexyl acrylate, 2-ethylhexyl methacrylate, or a mixture thereof. Alternatively or additionally, comonomer group C) includes or is 2-hydroxyethyl acrylate, 2-hydroxyethyl methacrylate, 4-hydroxybutyl acrylate, 4-hydroxybutyl methacrylate, or a mixture thereof. Alternatively or additionally, comonomer group D) includes or is N-benzyl-N-isopropylacrylamide, N-benzyl-N-isopropylmethacrylamide, N-benzyl-N-butylacrylamide, N-benzyl-N-butylmethacrylamide, N-benzyl-N-isobutylacrylamide, N-benzyl-N-isobutylmethacrylamide, N-benzyl-N-isopentylacrylamide, N-benzyl-N-isopentylmethacrylamide, N-benzyl-N-pentylacrylamide, N-benzyl-N-pentylmethacrylamide, N-benzyl-N-methylacrylamide, N-benzyl-N-methylmethacrylamide, or a mixture thereof. Alternatively or additionally, comonomer group E) includes or is N,N-dibenzyl(meth)acrylamide and N,N-diisopropyl(meth)acrylamide, N,N-dibenzyl(meth)acrylamide and N,N-dibutyl(meth)acrylamide, N,N-dibenzyl(meth)acrylamide and N,N-isobutyl(meth)acrylamide, N,N-dibenzyl(meth)acrylamide, N,N-diisopentyl(meth)acrylamide and N,N-dipentyl(meth)acrylamide, N,N-dibenzyl(meth)acrylamide and N,N-dimethyl(meth)acrylamide, or a mixture thereof. Alternatively or additionally, F) includes or is a crosslinker including at least two acrylate groups, at least two methacrylate groups, or at least one acrylate and at least one methacrylate group, where the crosslinker in some embodiments includes or is butane-1,4-diol diacrylate and/or ethylene glycol dimethacrylate. Such an ophthalmological composition is suitable for the production of clear ophthalmic biomaterial having an advantageous refractive index in the hydrated state nD, 35° C. of about 1.50 or more, a Shore A hardness of less than 80 (t=3 s) or a Shore A hardness of less than 50 (t=10 min), a glass transition temperature between 0° C. and 15° C., a water absorption capacity at 35° C. between 0.5% by weight and 3.5% by weight, and an Abbe number of at least 30.

With regard to the Shore A hardness values, it is to be noted that these are in some embodiments determined within the scope of the present disclosure at different indentation times t. The indentation times t are each included within the scope of the present disclosure. Normally, the measurement of an elastomer (for example of rubber) is effected after 3 seconds, as defined in DIN ISO 7619-1. The DIN-compliant Shore A limit for the materials of the described compositions is in some embodiments at a maximum of 80 or less (t=3 s), or in some embodiments at a maximum of 65 or less (t=3 s). Alternatively or additionally, the Shore A value is at most 50 or less with an indentation time t=10 min.

Further advantages arise in that the described ophthalmological composition contains at least one further component G), H), I), or a mixture thereof, where G) denotes at least one bindable UV absorber, in some embodiments a covalently bindable UV absorber, H) denotes at least one bindable dye, in some embodiments a covalently bindable dye, for modifying light absorption properties, and I) denotes a polymerization initiator. With the aid of component G), it is in some embodiments possible to provide UV absorption properties at least in the wavelength range between about 300 nm and about 400 nm. With the aid of component H), which is also referred to as yellow dye, the ophthalmological composition is adapted to provide a yellow biomaterial for production of lenses that has its absorption maximum for example in the wavelength range between about 400 nm and about 500 nm. The amount of the yellow dye used in the described compositions is in some embodiments chosen as required within a relatively wide concentration range in order to achieve a desired percentage transmission at each wavelength in the range between about 400 nm and about 500 nm. It is likewise possible by means of one or more of components G) and/or H), which are in some embodiments covalently bound in the reacted polymer in order to avoid outer diffusion, to achieve a desired absorption profile of the biomaterial or a lens produced therefrom within the wavelength range visible to man. With the aid of a polymerization initiator, it is possible to adjust the nature and speed of the polymerization reaction of the ophthalmological composition.

Further advantages with regard to various properties of the ophthalmological composition and of a biomaterial formed therefrom arise from, based on the total weight of the ophthalmological composition, a proportion of component G) of not more than 2% by weight, that is, for example, 0.1% by weight, 0.2% by weight, 0.3% by weight, 0.4% by weight, 0.5% by weight, 0.6% by weight, 0.7% by weight, 0.8% by weight, 0.9% by weight, 1.0% by weight, 1.1% by weight, 1.2% by weight, 1.3% by weight, 1.4% by weight, 1.5% by weight, 1.6% by weight, 1.7% by weight, 1.8% by weight, 1.9% by weight or 2.0% by weight, or in some embodiments not more than 1% by weight, and/or of component H) of not more than 5% by weight, that is, for example, 0.1% by weight, 0.2% by weight, 0.3% by weight, 0.4% by weight, 0.5% by weight, 0.6% by weight, 0.7% by weight, 0.8% by weight, 0.9% by weight, 1.0% by weight, 1.1% by weight, 1.2% by weight, 1.3% by weight, 1.4% by weight, 1.5% by weight, 1.6% by weight, 1.7% by weight, 1.8% by weight, 1.9% by weight, 2.0% by weight, 2.1% by weight, 2.2% by weight, 2.3% by weight, 2.4% by weight, 2.5% by weight, 2.6% by weight, 2.7% by weight, 2.8% by weight, 2.9% by weight, 3.0% by weight, 3.1% by weight, 3.2% by weight, 3.3% by weight, 3.4% by weight, 3.5% by weight, 3.6% by weight, 3.7% by weight, 3.8% by weight, 3.9% by weight, 4.0% by weight, 4.1% by weight, 4.2% by weight, 4.3% by weight, 4.4% by weight, 4.5% by weight, 4.6% by weight, 4.7% by weight, 4.8% by weight, 4.9% by weight or 5.0% by weight, and/or of component 1) of not more than 3% by weight, that is, for example, 0.1% by weight, 0.2% by weight, 0.3% by weight, 0.4% by weight, 0.5% by weight, 0.6% by weight, 0.7% by weight, 0.8% by weight, 0.9% by weight, 1.0% by weight, 1.1% by weight, 1.2% by weight, 1.3% by weight, 1.4% by weight, 1.5% by weight, 1.6% by weight, 1.7% by weight, 1.8% by weight, 1.9% by weight, 2.0% by weight, 2.1% by weight, 2.2% by weight, 2.3% by weight, 2.4% by weight, 2.5% by weight, 2.6% by weight, 2.7% by weight, 2.8% by weight, 2.9% by weight, or 3.0% by weight.

It has also been found to be advantageous in certain embodiments when the UV absorber G) is 2-(5-chloro-2H-benzotriazol-2-yl)-6-(1,1-dimethylethyl)-4 ethenylphenol (UVAM). Alternatively or additionally, it has been found to be advantageous in certain embodiments when the dye H) is the yellow dye 4-(3-vinylphenylazo)diphenylamine (3VPADPA or VPAD). Since the yellow dye absorbs in the range between 400 nm and 500 nm, it is also referred to as a “blue light absorber.” These compounds, individually or in any combination, allow the production of a biomaterial with an absorption profile that is particularly advantageous for ophthalmological lenses within the wavelength range between about 300 nm and 400 nm (G)) or between about 400 nm and about 500 nm (H)). In other words, a combination of UV and blue light absorbers (G), H)) is in certain embodiments used to adjust the absorption profile within the specified wavelength range as desired. The level of high-energy blue light is reduced in this way. If no blue light absorber (H)) is added, and only the UV absorber (G)) is used, the cut-off of the absorption is preferably set at about 400 nm and the biomaterial described herein remains colorless with maximum UV protection.

A second aspect of the compositions described herein relates to an ophthalmological lens made at least partly from the ophthalmological compositions described herein. The ophthalmological lens is in a particular embodiment a soft intraocular lens. The lens, in some embodiments, has an optical part and a tactile part. The lens, in some embodiments, also consists of two or more different biomaterials, of which at least one biomaterial is in accordance with the compositions described herein. The entire lens is in some embodiments produced from one or more compositions described herein or biomaterials described herein. The lens therefore, in some embodiments, has two or more areas with different optical, physical and/or mechanical properties. However, in some embodiments the ophthalmological lens is designed as a contact lens. Through the use of the ophthalmological compositions described herein for realization of the ophthalmological lens described herein, the lenses described herein are flexible, biocompatible, and have a high refractive index under physiological conditions with a particularly low risk of glistening. Furthermore, the ophthalmological lens described herein is stored dry and sterilized by steam sterilization in some embodiments. Further resultant features and the advantages thereof are inferrable from these descriptions.

In an one embodiment of the ophthalmological lens described herein, the ophthalmological lens in a non-hydrated state has a refractive index n_{D,20° C.}>1.51 and/or in a hydrated state has a refractive index n_{D,35° C.}>1.50 and/or a Shore A hardness less than 80 (t=3 s), or not more than 65 (t=3 s), or a Shore A hardness of less than 50 (t=10 min), and/or a glass transition temperature between 0° C. and 15° C., or between 4° C. and 9° C., and/or a water absorption capacity at 35° C. between 0.5% by weight and 3.5% by weight, or between 1.5% by weight and 2.5% by weight, and/or an Abbe number of at least 30, or at least 40. In this way, the ophthalmological lens described herein incorporates one or more particularly advantageous properties. The ophthalmological lens preferably fulfills all the above-mentioned properties.

In a further embodiment, the ophthalmological lens, in an in vitro glistening test by accelerated aging, in which the lens is first placed in a saline solution at 45° C.±1° C. for 24 h and then at 37° C.±1° C. for 2.5 h, has a microvacuole density of not more than 10 MVs/mm², or not more than 1 MVs/mm². In other words, the ophthalmological lens advantageously has a particularly low microvacuole density, where the microvacuole density is determined by the cited test method. The saline solution is, in some embodiments, a physiological saline solution (NaCl concentration 9 g/l, osmolarity 308 mOsm/l). In this way, problems that are otherwise caused by glistening, for example glare symptoms when driving at night with oncoming light or on a sunny day, are avoided completely or at least substantially completely. In a further embodiment, the lens is glistening-free and in particular microvacuole-free, which is achievable without difficulty because of the use of the ophthalmological composition described herein.

In a further embodiment, the ophthalmological lens is steam-sterilized. In this way, the lens is inexpensively sterilized by established steam sterilization, which advantageously makes it possible to dispense with chemical sterilization methods. Alternatively or additionally, the ophthalmological lens is plasma-treated. This reduces surface tack. For example, the plasma treatment is conducted in a furnace with an HF plasma generator (for example, 13.56 MHz) and in a mixed atmosphere of oxygen and argon (for example, 23 sccm 02, 127 sccm Ar, 400 mtorr) at 100 W to 800 W, or at about 400 W for 1 to 10 minutes, or for about 5 minutes per IOL side. The performance and duration can be varied as required to achieve the desired surface properties. In the plasma furnace, the oxygen is converted to ozone, which intensifies the treatment. Alternatively or additionally, the ophthalmological lens is stored in a non-hydrated state in a storage cartridge and/or in an implantation tool for implantation of the lens into an eye. Alternatively or additionally, the lens is advantageously used for dry and optionally fully preloaded injection systems and does not have to be packaged or stored in liquid. This simplifies packaging and significantly extends shelf life and handling.

A polymeric biomaterial produced by polymerization from the ophthalmological composition constitutes a further independent aspect of described compositions. In some embodiments, thermally induced free-radical polymerization is used. Likewise, in some embodiments photochemically induced polymerization reactions are used.

Other embodiments encompass plasma treatment of the described ophthalmological lens. This reduces the surface tack of the lens without the need for a chemical coating (for example, heparin in the immersion process). In one embodiment of the procedure of the plasma treatment and parameters of the plasma treatment are taken from the above description.

Further features of the described compositions and lenses made therefrom will be apparent from the claims and the working examples. The features and combinations of features mentioned in the description above and the features and combinations of features mentioned in the working examples below and/or shown on their own are contemplated as being usable not only in the respectively specified combination but also in other combinations or on their own. Contemplated and described herein are also configurations that are not shown and elucidated explicitly in the working examples but are apparent from and can be created through separate combinations of features from the configurations elucidated herein. The present disclosure shall also be considered to extend to embodiments and combinations of features that thus do not have all the features of an independent claim as originally worded.

### BRIEF DESCRIPTION OF DRAWINGS

The invention will now be described with reference to the drawings wherein:

FIG. 1 shows a schematic view of an ophthalmological lens according to one working example of the invention.

### DETAILED DESCRIPTION

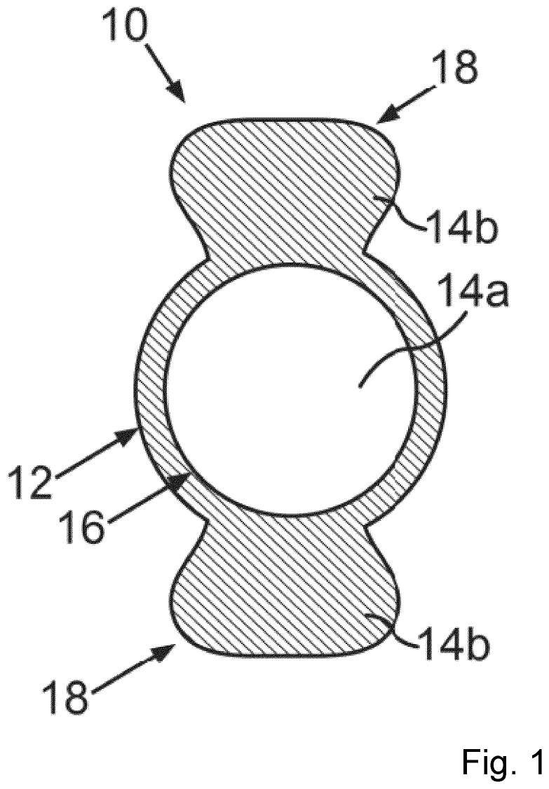

FIG. 1 shows a schematic view of an ophthalmological lens 10 according to one working example. The ophthalmological lens 10 in the present context takes the form of a soft intraocular lens (IOL) and has a main body 12. In the present example, the main body 12 has a first region 14a and a second region 14b, which differ with regard to at least one parameter from the group of stiffness and hardness. For this purpose, the regions 14a, 14b, for example, are produced from different embodiments of an ophthalmological composition discussed in detail below or subjected to different aftertreatment. In the simplest form, in one embodiment, however, the regions 14a, 14b are produced from the same ophthalmological composition and do not differ at the molecular level. The IOL 10 in the embodiment shown has an optical part 16 and a fundamentally optional tactile part 18, which is wing-shaped in the present case. However, the geometry of the tactile part 18 can and will in some cases fundamentally vary and take the form of a hook, for example.

For production of the IOL 10, an ophthalmological composition described herein was used, including comonomer groups A) to C), at least one comonomer group which is D), E) or a mixture thereof, and at least one crosslinker F). In this context, A) denotes at least one (meth)acrylate including at least one aromatic group; B) denotes at least one (meth)acrylate including an aliphatic or nonaromatic cyclic or nonaromatic heterocyclic group; C) denotes at least one (meth)acrylate including at least one hydroxy group; D) denotes at least one (meth)acrylamide including an aromatic group and including an aliphatic or nonaromatic cyclic or nonaromatic heterocyclic group, and E) denotes a mixture of at least one (meth)acrylamide including two aromatic groups and at least one (meth)acrylamide including two aliphatic and/or nonaromatic cyclic and/or nonaromatic heterocyclic groups.

The comonomer groups A) to D)/E) are discussed in detail in table 2 below together with their meaning, with illustrative compounds and with illustrative proportions by mass in the total weight of the composition. These proportions by mass relate to the sum total of all compounds of the respective class A) to D)/E). For example, if class A) includes three different compounds and has a proportion by mass of 50% by weight of the total weight of the composition, this proportion by mass is formed from the sum total of the respective proportions by mass of the three compounds of class A).

TABLE 2
Comonomer groups A) to D)/E)

| | Aromatic | Aliphatic | Hygroscopic | (Hybrid) acrylamide |
|---|---|---|---|---|
| | comonomers A) | comonomers B) | comonomers C) | comonomers D)/E) |
| Main function | Increases | Increases flexibility | Increases water | Increases structural |
| | refractive index | | absorption | strength |
| Side effects | Reduces flexibility | Reduces refractive | Reduces refractive | Increases glass |
| | and water | index and material | index | transition temperature |
| | absorption | thickness | | |
| Example | PEMA, PEA, | nBuA, iBuA, EHA | HEMA, HEA, HBA | BIPA, BBA, BIBA, BMA, |
| compounds | EGPEA | | | BHEA |
| Proportion | 39-50% | 22-36% | 13-19% | 3-11% |
| by mass | | | | |

The abbreviations mean:
PEMA 2-phenylethyl methacrylate
PEA 2-phenylethyl acrylate
EGPEA ethylene glycol phenyl ether acrylate
nBuA butyl acrylate
iBuA isobutyl acrylate
EHA 2-ethylhexyl acrylate
HEMA 2-hydroxyethyl methacrylate
HEA 2-hydroxyethyl acrylate
HBA 4 hydroxybutyl acrylate
BIPA N-benzyl-N-isopropylacrylamide
BBA N-benzyl-N-butylacrylamide
BIBA N-benzyl-N-isobutylacrylamide
BIPEA N-benzyl-N-isopentylacrylamide
BMA N-benzyl-N-methylacrylamide
BHEA N-benzyl-N-2-hydroxyethylacrylamide

As already mentioned, acrylates and corresponding methacrylates, and also acrylamides and corresponding methacrylamides, generally can be exchanged for or mixed with one another. Within the scope of the present disclosure, alkyl group substituents, in some embodiments, generally have from 1 to 12 carbon atoms. For example, it is generally possible to use, as (meth)acrylamide, a compound of the general formula III:

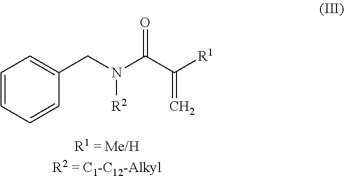

where the alkyl radical R² is in some embodiments unbranched or branched and unsubstituted or substituted by one, two, or more hydroxy group(s). When the alkyl radical R² is substituted by one or more hydroxy groups, at least one hydroxy group is terminal. The compound of the formula III is in some instances N-benzyl-N-isopropylacrylamide (BIPA):

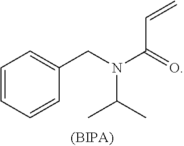

The compound of the formula III may be N-benzyl-N-isobutylacrylamide (BIBA):

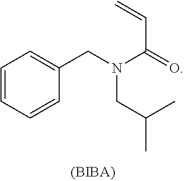 [SMILES: C=CC(=O)N(Cc1ccccc1)CC(C)C]

The compound of the formula III may be N-benzyl-N-isopentylacrylamide (BIPEA):

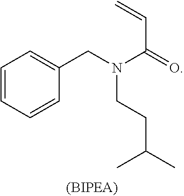 [SMILES: C=CC(=O)N(CCC(C)C)Cc1ccccc1]

The compound of the formula III may be N-benzyl-N-2-hydroxyethylacrylamide (BHEA):

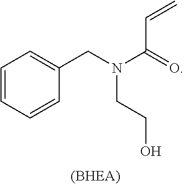

In this case, the ethyl radical R² is substituted by a terminal hydroxy group, such that the compound BHEA can interact with the comonomer C), and also with water molecules. In general, in some embodiments, two or more, for example two, hydroxy groups are provided as substituents, and so the compound of group D) conforms to the general formula IV in which n and m are respectively selected in the range from 1 to 10, so that the sum of n+m is in the range from 2 to 11.

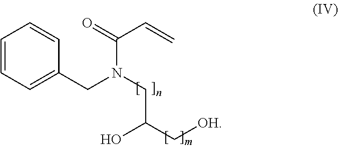

Instead of the acrylamides shown, as already mentioned, also contemplated are corresponding methacrylamides and any mixtures thereof.

The advantageous interactions of the different comonomer groups A) to D) (or E)) are explained in detail in the compositions V and VI that follow, with reference to the respective illustrative compound.

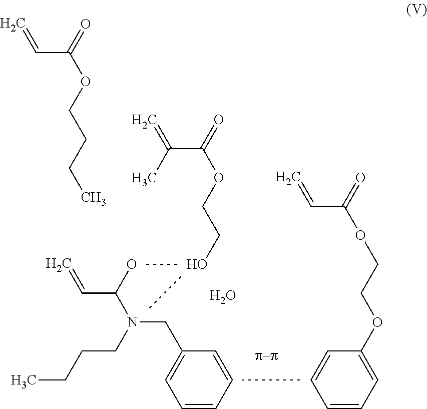

In composition V, the compounds EGPEA (comonomer type A)), nBuA (comonomer type B)), 2-HEMA (comonomer type C), and BBA (comonomer type D)) are used by way of example. In this example, in composition V, by virtue of the selected structural similarities of the individual comonomer types A) to D) (or similarly E)), advantageous intermolecular interactions are formed, in that the hybrid (meth)acrylamide D) acts as a kind of mediator between groups A) and B). The aryl group of the (meth)acrylamide D) also advantageously leads to an increase in refractive index and forms n-T interactions with the aryl groups of the aromatic monomer A), which leads to a higher structural strength of the polymer. The alkyl group of the (meth)acrylamide D) increases the flexibility of the material. Comonomer type C), on the other hand, by means of its terminal and sterically unhindered hydroxy group, can form hydrogen bonds to water molecules enclosed in the polymer and to the amide group of the (meth)acrylamide D)/E).

The same considerations apply to compositions VI, in which the compounds PEA (comonomer type A)), iBuA (comonomer type B)), 2-HEMA (comonomer type C)), and BIPA (comonomer type D)) are used by way of example:

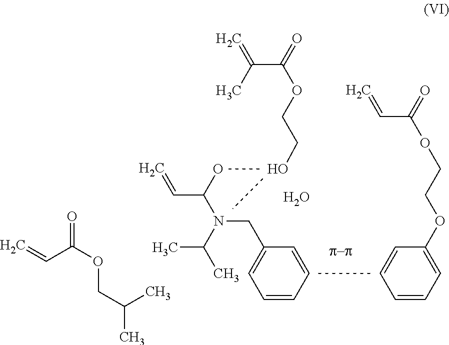

In accordance with the material design strategy described above, several novel ophthalmological compositions were produced, polymerized, and analyzed by way of example. The resulting polymeric biomaterials showed promising properties in terms of material flexibility and tensile strength, and also high refractive indices. In addition to the introduced monomers, (2-(5-chloro-2H-benzotriazol-2-yl)-6-(1,1-dimethylethyl)-4-ethenylphenol) (UVAM) as UV blocker for ophthalmic materials, 2,2′-azobis(2-methylpropionitrile) (AIBN) is used for thermal free-radical initiation of the composition. An illustrative ophthalmological composition, which is identified as “T-30C” and is present in the polymerized state as an optically clear biomaterial, is given in table 3 with the respective substance groups and amounts used.

TABLE 3
Inventive ophthalmological composition T-30C

| Group | Abbreviation | Name | [CAS] | Amount [g] | % by wt. |
|---|---|---|---|---|---|
| D) | BIPA | N-benzyl-N-isopropylacrylamide | 124762-00-1 | 40.32 | 4.6 |
| G) | UVAM | 2-(5-chloro-2H-benzotriazol-2-yl)- | 124883-10-9 | 6.83 | 0.8 |
| | | 6-(1,1-dimethylethyl)-4- | | | |
| | | ethenylphenol | | | |
| I) | AIBN | 2,2′-azobis(2-methylpropionitrile) | 78-67-1 | 0.97 | 0.1 |
| A) | PEA | 2-phenylethyl acrylate | 3530-36-7 | 338.67 | 38.9 |
| F) | BDA | butane-1,4-diol diacrylate | 1070-70-8 | 26.06 | 3.0 |
| C) | HEMA | hydroxyethyl methacrylate | 868-77-9 | 146.76 | 16.9 |
| B) | iso-BuA | isobutyl acrylate | 106-63-8 | 241.91 | 27.8 |
| B) | n-BuA | n-butyl acrylate | 141-32-2 | 68.54 | 7.9 |
| | | T-30C | | 870.06 | 100.0 |

However, the ophthalmological composition is suitable not only for the production of clear ophthalmic biomaterials. By addition of an appropriate amount (typically <1.0% by weight) of yellow dye (group H)), the ophthalmological composition is easily adaptable to produce a yellow biomaterial. The amount of yellow dye used in the composition is selected within a wide concentration range, as required. In this example, it was adjusted such that the composition had the same optical properties in terms of percentage transmission at each wavelength in the range from 400 nm to 500 nm as the commercially available yellow biomaterial ZEISS CT Lucia (Acrylmex Y). This was achieved by adding a small amount (<1.0% by weight) of the yellow dye 4-(3-vinylphenylazo)diphenylamine (3VPADPA or VPAD), which absorbs blue light, to the composition “T-30C” shown above. The correspondingly adjusted composition is referred to hereinafter as “T-30Y.”

Measurements on three clear (T-30C) and three yellow (T-30Y) biomaterial batches were used to determine the average values for the respective physical properties. An overview of the average properties of the biomaterials is given in table 4 for the clear version T-30C of the ophthalmological composition and in table 5 for the yellow version T-30Y of the ophthalmological composition.

TABLE 4
Properties of the optically clear material T-30C

| Category | Biomaterial properties | Value |
|---|---|---|
| Optical | wavelength (10% T), IOL (20 D)/nm | 402.0 |
| | Transmittance (550 nm)/% | >85.0 |
| | Refractive index (n_{D}) at 20° C., dry | 1.5160 |
| | Refractive index (n_{D}) at 35° C., hydrated | 1.5093 |
| | Abbe number at 35° C., hydrated | 43.5 |
| Physical | Glass transition temperature (T_{g})/° C. | 6.0 |
| | Water absorption capacity at 35° C./% by | 1.8 |
| | weight | |
| | Leachable substances after extraction/% | <0.2 |
| Mechanical | Hardness (analogous to Shore A, 10 min) | 43.7 |

TABLE 5
Properties of the yellow material T-30Y

| Category | Biomaterial properties | Value |
|---|---|---|
| Optical | wavelength (10% T), IOL (20 D)/nm | >400.0 |
| | Transmittance (550 nm)/% | >82.0 |
| | Refractive index (n_{D}) at 20° C., dry | 1.5159 |
| | Refractive index (n_{D}) at 35° C., hydrated | 1.5092 |
| | Abbe number at 35° C., hydrated | 42.3 |
| Physical | Glass transition temperature (T_{g})/° C. | 5.0 |
| | Water absorption capacity at 35° C./% by | 1.8 |
| | weight | |
| | Leachable substances after extraction/% | <0.2 |
| Mechanical | Hardness (analogous to Shore A, 10 min) | 44.2 |

In addition to the properties highlighted in table 4 and table 5, the biomaterial also has excellent resistance to glistening. The type and amount of crosslinker which is used for an elastomer affects the mechanical properties and is also, in some instances, used to reduce glistening. However, the change in the amount of crosslinker alone is not sufficient even in the case of compositions known from the prior art to achieve all the above-mentioned requirements on the material including the reduction in glistening. However, it was found that, even with a constant amount of crosslinker (for example, about 3% by weight), the addition of (meth)acrylamide comonomers D)/E) contributes to steering the material properties in the desired direction.

The ophthalmological compositions described herein, in contrast to the low-glistening hydrophilic compositions known from the prior art that have to be stored in saline solution, offer both the possibility of dry storage and the possibility of steam sterilization. The water absorbency of the polymerized biomaterial is tailored so that these conflicting requirements are achievable with just one single material.

For experimental confirmation, a glistening study was conducted. In the course of accelerated aging, five IOLs with a refractive force each of 20.0 D were produced from an ophthalmological composition as described herein and stored in 0.9% sodium chloride solution at a temperature of 45° C. for 24 hours, before the lenses were incubated at 37° C. for 2.5 hours and examined under a microscope with a digital camera and vacuole detection software (Image J). It was found that two of the five IOLs remained completely free of microvacuoles (MV) during the accelerated aging process. The three other IOLs had a very low microvacuole count of about 0.61 MV/mm². This density not only corresponds to grade 0 on the Miyata scale, but is also well below the MV density achievable by the vast majority of currently commercially available IOL models in previous studies. The results of these previous studies are summarized in table 6.

TABLE 6
Summary of the results of glistening studies of various IOL models

| | IOL model/ | Water | | |
|---|---|---|---|---|
| Manufacturer | biomaterial | content % | MV/mm² | Ø MV/mm² |
| Zeiss | T-30C | 1.8 | 0-0.61 | 0.37 |
| Kowa | Avansee | 2.0 | 0-1.00 | <1.00 |
| Alcon | Clareon | 1.5 | unknown | 4.09 |
| B&L | enVista | 4.0 | 3-6 | ~5 |
| Hoya | Vivinex | | 5.9-17.3 | 11.6 |
| | iSert XY1 | | | |
| J&J | Tecnis | | 12-36 | ~25 |
| Alcon | AcrySofSN60WF | | 7-266 | 40 |
| Alcon | AcrySofSN60WF | | 23-104 | 61 |
| Alcon | AcrySofSB30AL | | 19-832 | 316 |
| Nidek | Aktis SP | | 113-615 | 352 |
| | NS-60YG | | | |
| Alcon | AcrySof SA60AT | | 52-2179 | ~518 |
| Alcon | AcrySofMA60AC | | 136-1312 | 542 |
| Alcon | AcrySof | | 46-3862 | ~1760 |
| Hoya | iSert 231 | | 3058-4061 | 3532 |
| | PY60AD | | | |
| Hoya | iSymm | | 2545-6495 | ~4470 |

On the basis of the test results, the material composition T-30C is described not only as glistening-free but also as being suitable for the production of microvacuole-free lenses. It is important to note that the ophthalmological composition described in table 3 is adjustable both with regard to the compounds used for the individual comonomer groups and with regard to the concentration ranges of the individual comonomer groups. Numerous alternative formulations of the described compositions are thus possible within the scope of the present disclosure, and these also have the advantageous properties of the material described herein.

It was found that a biomaterial produced from the described compositions with a water absorption capacity of ˜1.8% by weight causes sufficient distribution of water in the polymer to show no glistening. At the same time, the water content is low enough not to cause problems by swelling of the lens after implantation, and so storage in an aqueous environment is unnecessary. In addition, the water content and chemical composition enable steam sterilization of the lenses.

It was determined that lenses with an optical zone of 5 mm, which were made of a composition described herein, for example from T-30C or T-30Y, is injectable without great expenditure of force by means of a Medicel Accuject injector with a 1.8 cartridge. This meets the requirements for a cataract operation with microincision. IOLs described herein were also injectable without difficulty with an Accuject 2.2 cartridge. Injection tests were performed in vitro using an OVD (ophthalmic viscoelastic device) or saline solution (BSS). The suitability results both from the high refractive index of the compositions described herein, which necessitates a lower lens thickness, and from its flexibility, which is strongly influenced by the low glass transition temperature (Tg) of the material. The latter should be designed very carefully, since too high a Tg can stiffen the polymer and too low a Tg can make the machining process more difficult. The described composition T-30C/T-30Y has a Tg of about 6° C. and is thus at the lower end of the range for hydrophobic acrylate-based IOL materials (cf. table 1). In addition to the production of lenses, mechanical tests were also conducted in order to demonstrate machinability of the biomaterial produced from the described compositions herein.

Furthermore, a T-30C biomaterial was characterized by dynamic-mechanical thermoanalysis (DMTA). In order to simulate the conditions during cryoturning and cryomilling of the materials, the respective average rotational speeds of turning (7500 rpm=125 Hz) and milling (14 000 rpm=233 Hz) were used as load frequencies. After the experimental analysis, shear storage modulus G′ and shear loss modulus G″ were determined. Several different noncovalent bonds can form between the functional polymer groups of the compositions described herein (cf. compositions V and VI). These forces are overcome when the biomaterial is heated (for example during steam sterilization) and automatically regenerate when the material is cooled. The DMTA tests showed that the Tg changes only about 2 K when the load frequency is doubled, which enables good stability throughout the machining process. In addition, the biomaterial included of the compositions described herein shows a significantly smaller decrease in material stiffness with increasing temperature in the vitreous state (shear storage modulus G′). Thus, when the biomaterial included of the compositions described herein is machined (for example at −20° C.), it therefore has higher stiffness. At room temperature or higher, T-30C/Y is distinctly softer, which, as already mentioned, leads to better implantability.

As well as the advantages already emphasized, these biomaterials were also tested for standard requirements for ophthalmic lenses. These tests include photostability studies, including the UV stability of the biomaterial, and the extraction of the storage solution. These tests were also successfully passed. In addition, a number of biocompatibility tests were conducted, including a cytotoxicity study and a risk analysis, which were also completed successfully. Tests for material tack have also been performed, which are particularly important since the biomaterials described herein have a low Tg and high flexibility. For this purpose, disks of biomaterial of the invention were cut with a smooth surface and pushed together with a defined force, before measuring the force required for detachment. In combination with additional injection tests, it was found that no chemical coating (for example heparin in the dipping process) is required for this biomaterial, and that a simple plasma treatment specifically developed for this purpose is sufficient to reduce surface tack. This plasma treatment process has also been shown to be stable over a long period of more than one year, ensuring safe unfolding of the lens after the storage period. Plasma treatment was performed in a furnace with an RF plasma generator (13.56 MHz) and in a mixed atmosphere of oxygen and argon (23 sccm 02, 127 sccm Ar, 400 mtorr) at 400 W and for 5 minutes per IOL side. In the plasma furnace, the oxygen is converted to ozone, which intensifies the treatment.

Finally, the biomaterials described herein were tested for their suitability for steam sterilization. For the tests, disks of thickness 1.0 mm and having a diameter of 6.0 mm were produced by cryogenic rotation from the biomaterial and from several hydrophobic acrylate materials known from the prior art. The disks were hydrated in an appropriate amount of aqueous solution in autoclavable vessels at room temperature for 48 hours. All vessels were transferred to an autoclave and heated to 121° C. at the standard rate (+6.6 K/min, 15 min in total). The vessels were kept at this temperature for 30 minutes before being cooled gradually to room temperature (21° C.). The cooling should preferably be effected at as slow a speed as possible (for example, −0.07 K/min, 24 h in total). The panes were then checked for visible defects, in particular microvacuoles, that have formed within the material. In the test conducted, the disks with the T-30C composition showed no permanent damage due to microvacuoles and were able to surpass the hydrophobic reference samples in this respect. Thus, the biomaterials and lenses described herein are suitable for steam sterilization.

Table 7 below reports further working examples of the ophthalmological compositions described herein. The biomaterials and lenses produced therefrom possess the same advantageous properties as the already discussed material T-30C/Y. The alternative compositions reported in table 7 follow the strategy described and show, by way of example and in a nonexhaustive manner, which variations are possible either by a comonomer exchange and/or by a change of the proportions of groups used in order to obtain comparable optical, physical and mechanical properties of the corresponding biomaterials.

TABLE 7
Working examples of the ophthalmological composition of the invention

| | | | | | | | Hardness | | | |
|---|---|---|---|---|---|---|---|---|---|---|
| | | | | | | | analogous | | | |
| | | | | | | Water | to Shore | 45° C. − | RI, | |
| | | | | Group | | content | A | 20° C. | nD | Tg |
| Designation | Group A) | Group B) | Group C) | D)/E) | Group F) | 35° C. | (10 min) | glistening | 35° C. | [° C.] |
| T-8 | 46% | 22% | 22% | 7% | 3.5% | 2.7% | 47.4 | — | 1.523 | 4 |
| | PEA | EHA | HEMA | BIPA | BDA | | | | | |
| T-10 | 48% | 23% | 17% | 7% | 3.5% | 2.1% | 46.2 | — | 1.525 | 9 |
| | PEA | nBuA | HEMA | BIPA | BDA | | | | | |
| T-12 | 46% | 22% | 16% | 11% | 3.5% | 2.1% | 45.7 | — | 1.527 | 9 |
| | PEA, EGPEA | EHA | HEMA | BIPA | BDA | | | | | |
| T-14 | 49% | 23% | 17% | 7% | 3.5% | 2.1% | 46.5 | — | 1.522 | 6 |
| | PEA, APEA | EHA | HEMA | BIPA | BDA | | | | | |
| T-16 | 49% | 23% | 17% | 7% | 3.5% | 2.1% | 43.8 | — | 1.525 | 7 |
| | PEA | nBuA | HEMA | BIPA | BDA | | | | | |
| T-17 | 50% | 24% | 13% | 10% | 3.0% | 2.0% | 42.3 | — | 1.526 | 8 |
| | PEA, EGPEA | EHA | HEMA | BIPA | BDA | | | | | |
| T-20 | 49% | 23% | 17% | 7% | 3.5% | 1.9% | 45.4 | — | 1.525 | 8 |
| | PEA | nBuA | HEMA | BIPA | BDA | | | | | |
| T-30 | 39% | 36% | 17% | 5% | 3.0% | 1.8% | 43.7 | — | 1.512 | 6 |
| | PEA | nBuA, | HEMA | BIPA | BDA | | | | | |
| | | iBuA | | | | | | | | |
| T-35 | 39% | 35% | 17% | 5% | 3.0% | 2.0% | 43.3 | — | 1.523 | 7 |
| | PEA | nBuA, | HEMA | BIPA | BDA | | | | | |
| | | iBuA | | | | | | | | |
| T-36 | 39% | 36% | 17% | 5% | 3.0% | 1.9% | 43.7 | — | 1.510 | 6 |
| | PEA | nBuA, | HEMA | BIPA | BDA | | | | | |
| | | iBuA | | | | | | | | |
| Min | 39% | 22% | 13% | 5% | 3.0% | 1.8% | 42.3 | — | 1.510 | 4 |
| Max | 50% | 36% | 22% | 11% | 3.5% | 2.7% | 47.4 | — | 1.527 | 9 |

The parameter values specified in the documents to define process and measurement conditions for the characterization of specific properties of the subject matter described herein should also be considered to be encompassed by the scope of the present description in the context of deviations—for example due to measurement errors, system errors, weighing errors, DIN tolerances and the like.

It is understood that the foregoing description is that of the preferred embodiments of the invention and that various changes and modifications may be made thereto without departing from the spirit and scope of the invention as defined in the appended claims.

### LIST OF REFERENCE SIGNS

10 lens (IOL)
12 main body
14a first region
14b second region
16 optical part
18 tactile part

1. An ophthalmological composition for production of an ophthalmological lens, comprising:
comonomer group A), comonomer group B), and comonomer group C),
at least one of comonomer group D) and comonomer group E), or a mixture thereof, and
at least one crosslinker F),
wherein:
the comonomer A) comprises at least one (meth)acrylate comprising at least one aromatic group;
the comonomer B) comprises at least one (meth)acrylate comprising an aliphatic or nonaromatic cyclic or nonaromatic heterocyclic group;
the comonomer C) comprises at least one (meth)acrylate comprising at least one hydroxy group;
the comonomer D) comprises at least one (meth)acrylamide comprising: (i) an aromatic group, and (ii) an aliphatic group, a nonaromatic cyclic group, or nonaromatic heterocyclic group; and
the comonomer E) comprises a mixture of: (a) at least one (meth)acrylamide comprising two aromatic groups, and (b) at least one (meth)acrylamide comprising two aliphatic groups, nonaromatic cyclic groups, and/or nonaromatic heterocyclic groups; and wherein based on the total weight of the ophthalmological composition:
a proportion of the comonomer group A) is between 30% and 60% by weight;
a proportion of the comonomer group B) is between 10% and 45% by weight;
a proportion of the comonomer group C) is between 5% and 30% by weight;
a proportion of the sum total of the comonomer group D) and the comonomer group E) is between 1% by weight and 14% by weight; and
a proportion of the crosslinker F) is not more than 5% by weight.

2. The ophthalmological composition as claimed in claim 1, wherein:
the comonomer group A) comprises or is 2-phenylethyl (meth)acrylate, ethylene glycol phenyl ether (meth)acrylate, or a mixture thereof;
the comonomer group B) comprises or is butyl (meth)acrylate, isobutyl (meth)acrylate, 2-ethylhexyl (meth)acrylate, or a mixture thereof;
the comonomer group C) comprises or is 2-hydroxyethyl (meth)acrylate, 4-hydroxybutyl (meth)acrylate, or a mixture thereof;
the comonomer group D) comprises or is N-benzyl-N-isopropyl(meth)acrylamide, N-benzyl-N-butyl(meth)acrylamide, N-benzyl-N-isobutyl(meth)acrylamide, N-benzyl-N-isopentyl(meth)acrylamide, N-benzyl-N-pentyl(meth)acrylamide, N-benzyl-N-methyl(meth)acrylamide, or a mixture thereof;
the comonomer group E) comprises or is N,N-dibenzyl(meth)acrylamide and N,N-diisopropyl(meth)acrylamide, N,N-dibenzyl(meth)acrylamide and N,N-dibutyl(meth)acrylamide, N,N-dibenzyl(meth)acrylamide and N,N-isobutyl(meth)acrylamide, N,N-dibenzyl(meth)acrylamide, N,N-diisopentyl(meth)acrylamide and N,N-dipentyl(meth)acrylamide, N,N-dibenzyl(meth)acrylamide and N,N-dimethyl(meth)acrylamide, or a mixture thereof; and/or
the crosslinker F) comprises at least two (meth)acrylate groups.

3. The ophthalmological composition as claimed in claim 1, further comprising at least component G), component H), component I) or a mixture thereof, wherein:
the component G) comprises at least one bindable UV absorber;
the component H) comprises at least one bindable dye; and
the component I) comprises a polymerization initiator.

4. The ophthalmological composition as claimed in claim 3, wherein, based on a total weight of the ophthalmological composition:
a proportion of the component G) is not more than 2% by weight;
a proportion of the component H) is not more than 5% by weight; and/or
a proportion of the component I) is not more than 3% by weight.

5. The ophthalmological composition as claimed in claim 3, wherein:
the component G) comprises or is 2-(5-chloro-2H-benzotriazol-2-yl)-6-(1,1-dimethylethyl)-4 ethenylphenol, and/or
wherein the component H) comprises or is 4-(3-vinylphenylazo)diphenylamine.

6. An ophthalmological lens, which is at least partly produced from the ophthalmological composition as claimed in claim 1.

7. The ophthalmological lens as claimed in claim 6, wherein the ophthalmological lens:
in a non-hydrated state has a refractive index n_{D,20° C.}>1.51;
in a hydrated state has a refractive index n_{D,35° C.}>1.50;
has a Shore hardness A of less than 80 (t=3 s) and/or a Shore hardness A of less than 50 (t=10 min);
has a glass transition temperature between 0° C. and 15° C.;
has a water absorption capacity at 35° C. between 0.5% by weight and 3.5% by weight;
has an Abbe number of at least 30, preferably at least 40.

8. The ophthalmological lens as claimed in claim 6, wherein the ophthalmological lens, in an in vitro glistening test by accelerated aging, in which the lens is first placed in a saline solution at 45° C.±1° C. for 24 hours and then at 37° C.±1° C. for 2.5 hours, has a microvacuole density of not more than 10 MVs/mm².

9. The ophthalmological lens as claimed in claim 6, wherein the ophthalmological lens has been steam-sterilized, plasma-treated, is stored in a storage cartridge, and/or in an implantation tool for implantation of the lens into an eye.

10. The ophthalmological composition as claimed in claim 1, wherein the crosslinker F) comprises or is butane-1,4-diol diacrylate and/or ethylene glycol dimethacrylate.

11. The ophthalmological composition as claimed in claim 3, wherein in the component G) the at least one bindable UV absorber is a covalently bindable UV absorber; and wherein in the component H) the at least one bindable dye is a covalently bindable dye.

12. The ophthalmological lens as claimed in claim 6, wherein the lens is a soft intraocular lens.

13. The ophthalmological lens as claimed in claim 8, wherein the lens has a glass transition temperature between 4° C. and 9° C.

14. The ophthalmological lens as claimed in claim 7, wherein the lens has a water absorption capacity at 35° C. between 1.5% by weight and 2.5% by weight.

15. The ophthalmological lens as claimed in claim 7, wherein the lens, in an in vitro glistening test by accelerated aging, in which the lens is first placed in a saline solution at 45° C.±1° C. for 24 hours and then at 37° C.±1° C. for 2.5 hours, has a microvacuole density of not more than 1 MVs/mm².

16. The ophthalmological lens as claimed in claim 6, wherein the ophthalmological lens has been steam-sterilized, plasma-treated, is stored in a non-hydrated state in a storage cartridge, and/or in an implantation tool for implantation of the lens into an eye.

17. The ophthalmological lens as claimed in claim 1, wherein the proportion of the crosslinker F) is between 0.5% by weight and 5% by weight.
